(19) United States
(12) Patent Application Publication
(10) Pub. No.: US 2025/0273331 A1
(43) Pub. Date: Aug. 28, 2025

(54) SYSTEM AND METHOD FOR PREDICTION OF TREATMENT DEVICE CHURN

(71) Applicant: Reciprocal Labs Corporation, Madison, WI (US)

(72) Inventors: Nicholas John HIRONS, Madison, WI (US); Luke Michael Doolittle, Madison, WI (US)

(21) Appl. No.: 18/247,525

(22) PCT Filed: Oct. 1, 2021
(86) PCT No.: PCT/US2021/053156

§ 371 Date: Mar. 31, 2023

Related U.S. Application Data
(60) Provisional application No. 63/086,924, filed on Oct. 2, 2020.

(51) Int. Cl. G16H 40/67; A61M 15/00; G16H 50/70

(52) U.S. Cl. CPC G16H 40/67; A61M 15/00; G16H 50/70; A61M 2205/18; A61M 2205/3592; A61M 2205/52

(57) ABSTRACT

A system and method to predict churn in relation to use of a treatment device by a patient is disclosed. A communication interface collects event data relating to operating the treatment device delivering medicament to the patient. The event data and clinical data of the patient is stored. A churn analysis module inputs the event data and clinical data and applies a machine learning model to determine the likelihood of churn for the patient over a predetermined period from the input event data and clinical data. The machine learning model is trained from a machine learning pipeline having inputs of event data and clinical data from a population of patients using the treatment device to determine at least one trigger for patient churn. It is determined whether the likelihood of churn is over a threshold value. An action relating to the patient is triggered if the likelihood of churn is over the threshold value.

### CROSS-REFERENCE TO RELATED APPLICATIONS

This application claims the benefit of, and priority to, U.S. Provisional Patent Application No. 63/086,924 filed Oct. 2, 2020, which is hereby incorporated by reference herein in its entirety.

### TECHNICAL FIELD

The present disclosure relates generally to methods of improving adherence to applications that run medical devices, and more specifically to determining and predicting factors influencing churn for users of a medication device.

### BACKGROUND

Respiratory ailments such as asthma and chronic obstructive pulmonary disease (COPD) remains a significant and costly public health problem. For example, in the United States, more than 22 million people have asthma. Worldwide, the World Health Organization estimates the population with asthma may be 300 million, and predicts that it will rise to 400 million by 2025. Similarly, recent studies by the Centers for Disease Control and Prevention have listed COPD as the third leading cause of death in the United States while estimating close to 13 million people may have COPD induced impaired lung function.

Despite the development of new medications, rates of hospitalizations and emergency room visits have not declined. Each year in the United States asthma causes approximately 2 million emergency department visits, 500,000 hospitalizations, and 5,000 deaths. In addition, asthma is responsible for an estimated 15 million missed days of school, and 12 million days of work. Total annual costs to US health insurers and employers are greater than $18 billion. COPD causes approximately 715,000 hospitalizations, and 134,000 deaths yearly. Additionally, the cost to the nation for COPD was recently projected to be approximately $49.9 billion, including $29.5 billion in direct health care expenditures, $8.0 billion in indirect morbidity costs and $12.4 billion in indirect mortality costs.

The majority of asthma exacerbations could be prevented with currently available treatments, however, only 1 in 5 asthmatics has the disease under control. Such treatments often rely on identifying a triggering condition of an asthma condition and properly administering treatment such as a medicament. One mechanism for a patient to self administer a medicament is an inhaler. When respiratory symptoms occur, a patient may administer the medicament via a puff from the inhaler.

Similarly, persons with COPD often carry an inhaled medication to immediately relieve symptoms whenever or wherever they occur. The frequency and location with which patients use these medications are important indicators of how well the disease is controlled and treated. Currently inhalers may include either a built in sensor or an attached sensor to gather information about use of the inhaler. This information may then be sent to a central server to determine adherence.

However, many patients do not adhere to the correct use of the inhaler and sensor combinations, resulting in a high churn rate. The lack of adherence means that patients do not properly receive the treatment for respiratory ailments. Many digital health applications and devices experience high rates of churn relative to other consumer applications and devices. Payors, providers and health care professionals currently have a limited ability to understand, predict and intervene when a user is likely to churn imminently in relation to treatment devices such as inhalers. The lack of adherence and high churn rate reduces the cost effectiveness of such treatments and the ability track and understand inhaler usage.

There is a need for a system that allows analysis to reduce high rates of churn for treatment devices. There is a need for a system that collects a variety of data to determine factors for determining the likelihood of churn for a patient that uses a treatment device. There is a further need for a system that determines patients with a high risk of churn to provide preventive measures and remedies for that patient.

### SUMMARY

The disclosed analysis system allows the curation of behavioral and static data for patients in order to train and deploy predictive models as to increasing adherence. The analysis allows analysis of the churn for a patient using a treatment device.

One disclosed example is a system to predict churn in relation to use of a treatment device by a patient. The system includes a communication interface to collect event data on monitoring the operation the treatment device. A storage device stores the event data and clinical data of the patient. A churn analysis module inputs the event data and clinical data and applies a machine learning model to determine the likelihood of churn for the patient over a predetermined period from the input event data and clinical data. The machine learning model is trained from a machine learning pipeline having inputs of event data and clinical data from a population of patients using the treatment device to determine at least one trigger for patient churn. The analysis module determines whether the likelihood of churn is over a threshold value. The module triggers an action relating to the patient if the likelihood of churn is over the threshold value.

A further implementation of the example system is an embodiment where the treatment device is a rescue inhaler or a control inhaler. Another implementation is where the communication interface communicates with a mobile device of the patient. A sensor in communication with the treatment device is synched with an application executed by the mobile device to provide event data. Another implementation is where the machine learning pipeline is trained from customer service data relating to the population of patients. Another implementation is where the action is one of a patient alert, a health care provider alert, or a treatment device manufacturer alert. Another implementation is where the machine learning pipeline responds with a prediction endpoint including the determined likelihood of churn, the values of the processed clinical data and the event data, and a quantified impact of how the values influence the determined likelihood of churn.

Another disclosed example is a method to determine churn of a treatment device operated by a patient. Clinical data of the patient is collected. Event data relating to monitoring the operation of the treatment device is collected via a communication interface. A machine learning model is applied to determine the likelihood of churn for the patient over a predetermined period from the input event data and clinical data. The machine learning model is trained from a machine learning pipeline having inputs of event data and clinical data from a population of patients using the treatment device to determine at least one trigger for patient churn. Whether the likelihood of churn is over a threshold value is determined. An action relating to the patient is triggered if the likelihood of churn is over the threshold value.

A further implementation of the example method is an embodiment where the treatment device is a rescue inhaler or a control inhaler. Another implementation is where the example method includes communicating with a mobile device of the patient via the communication interface; and synching a sensor in communication with the treatment device with an application executed by the mobile device to provide the event data. Another implementation is where the machine learning pipeline is trained from customer service data relating to the population of patients. Another implementation is where the action is one of a patient alert, a health care provider alert, or a treatment device manufacturer alert. Another implementation is where the machine learning pipeline responds with a prediction endpoint including the determined likelihood of churn, the values of the processed clinical data and the event data, and a quantified impact of how the values influence the determined likelihood of churn.

Another disclosed example is a pipeline method of training a machine learning model for predicting the churn of a patient operating a treatment device. Data inputs are collected from a population of patients. The data inputs include clinical data and event data relating monitoring the operation of the treatment device. The data inputs are curated to produce a data table. The machine learning model is trained from the data table to weight a plurality of input factors affecting churn and determine the impact of the input factors in determining a likelihood of churn to produce a prediction endpoint. The trained machine learning model is stored.

A further implementation of the example method includes refining the data table to exclude data relating to patients with insufficient interaction with the treatment device via a preprocessing module; and converting data for input to the training module is an embodiment where the treatment device is a rescue inhaler or a control inhaler. Another implementation is where the data inputs include data from mobile devices operated by the population of patients, data from an application executed on the mobile device, the application interfacing with the treatment device, and customer service data. Another implementation is where the stored model is executed by a prediction endpoint module to provide a prediction of the likelihood of churn relating to a single patient based on clinical data and event data of the treatment device by the single patient.

Another disclosed example is a machine learning pipeline system for training a machine learning model for predicting the churn of a patient operating a treatment device. A data input interface collects data inputs from a population of patients. The data inputs include clinical data and event data relating to monitoring the operation of the treatment device. A curation module curates the data inputs and produces a data table. A training module trains the machine learning model from the data table to weight a plurality of input factors affecting churn and determine the impact of the input factors in determining a likelihood of churn to produce a prediction endpoint. A storage device stores the trained machine learning model.

A further implementation of the example machine learning pipeline system is an embodiment including a preprocessing module refining the data table to exclude data relating to patients with insufficient interaction with the treatment device and converting data for input to the training module where the treatment device is a rescue inhaler or a control inhaler. Another implementation is where the data inputs include data from mobile devices operated by the population of patients, data from an application executed on the mobile device, the application interfacing with the treatment device, and customer service data. Another implementation is where the stored model is executed by a prediction endpoint module to provide a prediction of the likelihood of churn relating to a single patient based on clinical data and event data of the treatment device by the single patient.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 shows a churn analytics system for providing predictions of churn for patients using treatment devices;

FIG. 2 is a high-level block diagram illustrating an example of a computing device used in either as a client device, application server, and/or database server;

FIG. 3 is a block diagram of an example machine learning pipeline;

FIG. 4 is an example of a curated data table produced by the example machine learning pipeline in FIG. 3;

FIG. 5 is a block diagram of the model training and development module of the machine learning pipeline in FIG. 3; and

FIG. 6 is a service endpoint produced by the machine learning pipeline in FIG. 3.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a system that collects relevant data for churn of a treatment device such as a medicament inhaler. Factors relating to churn rate are determined from the gathered data via a machine learning pipeline.

One benefit of the churn analysis is improvements in the treatment devices as the analysis enables product and engineering teams to design features that address situations and behaviors that are associated with future churn. Another benefit is that the identified factors relating to churn may be applied to a more personalized communications, content, and features for individual patients to reduce churn rate. The ability to predict when the probability a user will churn in the next predetermined time such as 30 days, and the factors that impact that probability, allows a proactive communication to susceptible patients. This allows a resolution of any issue ahead of time, or offer certain incentives for behavioral change, leading to stronger retention and therefore better long term clinical outcomes. The better adherence to personal treatment devices such as inhalers may reduce emergency visits and hospitalization by a significant amount over a 12 month period. Improving patient retention with this service is therefore highly likely to reduce healthcare utilization. The analysis allows an improvement in adherence by a significant margin. Retaining patients therefore lead to increased fills of medications for pharma partners. Those medications are in turn proven to improve clinical outcomes.

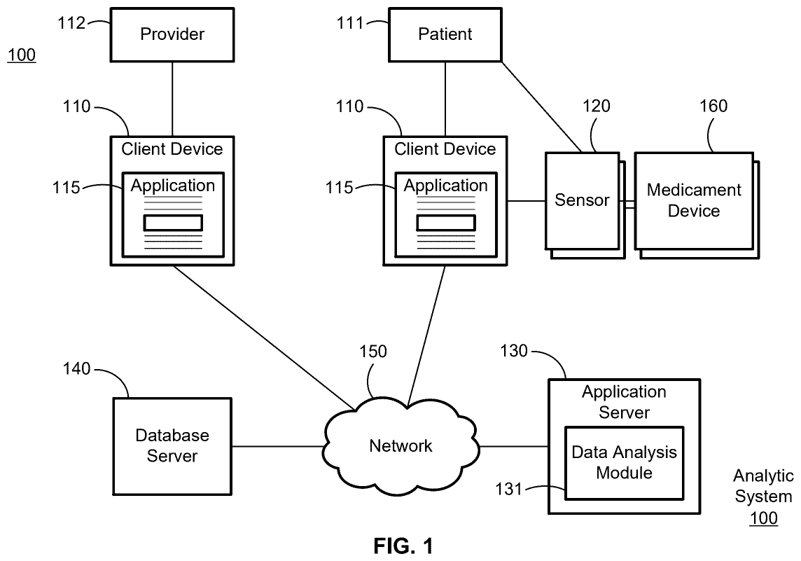

FIG. 1 shows a churn analytics system 100 for collecting data based on monitoring accurate, real-time medicament device events, performing analytics on that data, and providing outputs based on identification of indications of a high churn of a specific treatment device for a general patient population, according to one embodiment.

The analytics system 100 includes client computing devices 110, a medicament device sensor 120, a medicament device 160, an application server 130, a database server 140, and a network 150. Although FIG. 1 illustrates only a single instance of most of the components of the analytics system 100, in practice more than one of each component may be present, and additional or fewer components may be used.

The client devices 110, at the behest of their users, interact with the analytics system 100 via the network 150. For purposes of explanation and clarity it is useful to identify at least two different types of users. A patient 111 is a user burdened with a respiratory ailment such as asthma or COPD who makes use of the respiratory ailment analytics system 100 at least in part to obtain personalized rescue event risk notifications provided by the server 130 and management notifications created by their health care provider 112 in this example. Such notifications can be provided in exchange for the user's permission to allow the respiratory ailment analytics system 100 to monitor the patient's 111 medicament device 160 usage. As will be explained below, medication events are detected by a sensor 120 associated with the medicament device 160 and the user's client device 110, which in turn reports to the application server 130. In this example, the patients 111 represent a patient population, for which individual data is taken for each patient in the group. Another type of user may use the medicament device 160 for control medication to control symptoms of respiratory ailment.

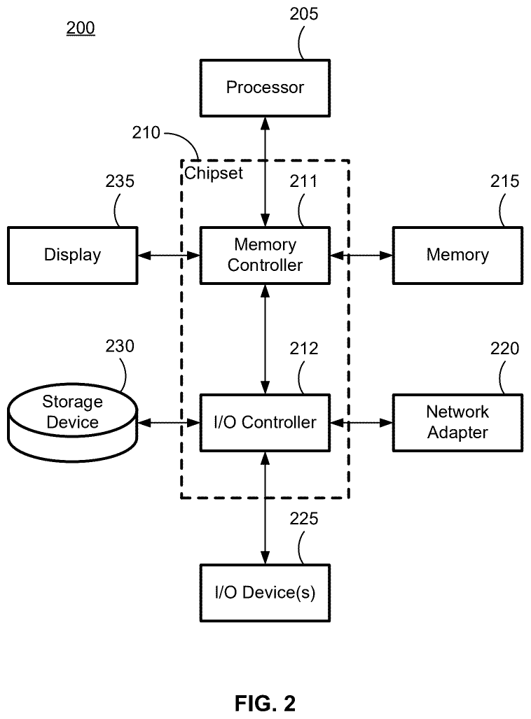

The client device 110 is a computer system. An example physical implementation is described more completely below with respect to FIG. 2. The client device 110 is configured to wirelessly communicate with the respiratory ailment analytics system 100 via network 150. With network 150 access, the client device 110 transmits to system 100 the user's geographical location and the time of a rescue or control medication event, as well as information describing the event as received from the associated medicament device sensor 120 (referred to throughout as “sensor 120”).

Regarding user location and event times, the client device 110 may determine the geographical location and time of a rescue or control event through use of information about the cellular or wireless network 150 to which it is connected. For example, the current geographical location of the client device 110 may be determined by directly querying the software stack providing the network 150 connection. Alternatively, the geographical location information may be obtained by pinging an external web service (not shown in FIG. 1) made accessible via network 150. The time of an event can be provided by the sensor 120 as part of the event data or added to event data by querying an appropriate software routine available as part of the client device's native operating system.

Application 115 provides a user interface (herein referred to as a “dashboard”) that is displayed on a screen of the client device 110 and allows a user to input commands to control the operation of the application 115. The dashboard is the mechanism by which healthcare providers 112 and patients 111 access the resources managed by the system 100. For example, the dashboard allows patients 111 and providers 112 to interact with each other, receive asthma rescue event risk notifications, exchange messages about treatment, provide and receive additional event and non-event data, and so on. The application 115 may be coded as a web page, series of web pages, or content otherwise coded to render within an internet browser. Application 115 may also be coded as a proprietary application configured to operate on the native operating system of the client device 110.

In addition to providing the dashboard, application 115 may also perform some data processing on rescue or control event data locally using the resources of client device 110 before sending the processed data through the network 150. Thus, in this example the application 115 allows communication of event data from the sensor 120 in relation to the operation of the medicament device 160. Event data sent through the network 150 is received by the application server 130 where it is analyzed and processed for storage and retrieval in conjunction with database server 140. The application server 130 may direct retrieval and storage request to the database server 140 as required by the client application 115. As will be explained, this analysis may be extended to include event data from multiple patients 111.

The client device 110 communicates with the sensor 120 using a network adapter and either a wired or wireless communication protocol, an example of which is the Bluetooth Low Energy (BTLE) protocol. BTLE is a short-ranged, low-powered, protocol standard that transmits data wirelessly over radio links in short range wireless networks. After the sensor 120 and client device 110 have been paired with each other using a BTLE passkey, the sensor 120 automatically synchronizes and communicates information relating to medicament device usage with the client device 110. If the sensor 120 hasn't been paired with a client device 110 prior to a rescue medication event, the information is stored locally until such a pairing occurs. Upon pairing, the sensor 120 communicates any stored event records to the client device 110. In other implementations, other types of wireless connections are used (e.g., infrared or IEEE 802.11).

Although client devices 110 and medicament devices 160 are described above as being separate physical devices (such as smart phones and inhalers, respectively), in the future it is contemplated the medicament devices 160 may include not only sensors 120 integrated into a single housing with the device 160, but also aspects of the client device 110. For example, a medicament device 160 may include an audiovisual interface including a display or other lighting elements as well as speakers for presenting visual audible information. In such an implementation the medicament device 160 itself may present the contents of notifications provided by the server 130 directly, in place of or in addition to presenting them through the client devices 110.

The example medicament device 160 is a medical device used to deliver medication to the lungs of a user experiencing a respiratory ailment rescue event. Different medicaments are used for asthma and COPD. Different medicaments may also be used for rescue and control for asthma and COPD. Medicament devices (e.g. inhalers) are typically portable and small enough to be carried by hand for ease of accessibility when treating respiratory symptoms. In one embodiment, medicine is delivered in aerosol form through a medicament device 160 such as a metered dose inhaler. Metered dose inhalers included a pressured, propellant canister of aerosol medicine, a metering valve for delivering a regulated medicine dosage amount, and a plastic holder that holds the pressurized canister and also forms a mouthpiece for delivery of the medicine. In another embodiment, medicine is delivered in dry powder form through a medicament device 160 such as a dry powder inhaler. Dry powder inhalers may have Cartesian ovular shaped bodies that house wheel and gear mechanisms enabling a user to index through a strip of dry powder medication. The bodies of dry powder inhalers also include a manifold and a mouthpiece to deliver dry powder to the user. Although a medicament device is used in this example, it is understood the churn analysis may be used for any personal treatment device that requires the user to use the device on a periodic basis. Further, although the above examples relate to respiratory disorders, the principles described herein may be applied to treatment regimes for other types of ailments.

Each patient may be associated with more than one medicament device 160. For example, the patient may have a rescue medicament device 160 that dispenses rescue medication, and a controller medicament device 160 that dispenses controller medication. Similarly, each patient may be associated with more than one sensor 120, each chosen to operate with one of the patient's medicament devices 160.

Generally, a sensor 120 is a physical device that monitors the usage of the medicament dispenser 160. The sensor 120 is either removably attachable to the medicament dispenser without impeding the operation of the medication dispenser, or the sensor 120 is an integrated component that is a native part of the medicament dispenser 160 as made available by its manufacturer.

The sensor 120 includes its own network adapter (not shown) that communicates with the client device 110 either through a wired connection, or more typically through a wireless radio frequency connection. In one embodiment, the network adapter is a Bluetooth Low Energy (BTLE) wireless transmitter, however in other embodiments other types of wireless communication may be used (e.g., infrared, IEEE 802.11).

The sensor 120 may also be configured to communicate more directly with the application server 130. For example, if the network adapter of the sensor 120 is configured to communicate via a wireless standard such as IEEE 802.11 or LTE, the adapter may exchange data with a wireless access point such as a wireless router, which may in turn communicate with the application server 130 without necessarily involving the client device 110 in every exchange of data. These two methods of communicating are not mutually exclusive, and the sensor 120 may be configured to communicate with both the client device 110 and the application server 130, for example using redundant transmission to ensure event data arrives at the application server 130 or to provide information directly to the client device 110 while the application server 130 is determining what notification to provide in response to an event.

As introduced above, the sensor 120 captures data about usage of the medicament device 160. Specifically, each sensor 120 captures the time and geographical location of the rescue medication event, that is, usages of the rescue medicament device 160, by the patient 111. Each sensor 120 transmits the event data in real-time or as soon as a network connection is achieved, automatically without input from the patient 111 or health care provider 112. The medication event information is sent to the application server 130 for use in analysis, generation of asthma rescue event notifications, and in aggregate analyses of event data across multiple patients for the purpose of determining churn.

To accomplish this goal, there are a number of different ways for the sensor 120 to be constructed, and in part the construction will depend upon the construction of the medicament device itself 160. Generally, all sensors 120 will include an onboard processor, persistent memory, and the network adapter mentioned above that together function to record, store, and report medication event information to the client device 110 and/or server 130. Sensors 120 may also include a clock for recording the time and date of events.

Regarding specific sensor 120 constructions, traditional inhalers, such as mechanical dose counters, are not designed with sensors 120 in mind, and thus the sensor 120 may be constructed accordingly. Some implementations in this manner include mechanical, electrical, or optical sensors to detect movement of the device 160, priming of the device, activation of the device, inhalation by the user, etc. In contrast, modern inhalers, such as deflectable membrane dose counters, include electrical circuitry may report event information as an electrical data signal which a sensor 120 is designed to receive and interpret, for example the medicament device 160 itself may report movement, priming, and activation to the sensor 120.

More information regarding hardware and software components for the sensors 120 and medicament devices 160, as well as the interaction between them to record rescue medication events can be found in U.S. patent application Ser. No. 12/348,424, filed Jan. 1, 2009, and International Application No. PCT/US2014/039014, filed May 21, 2014, both of which are incorporated by reference herein in their entirety.

The application server 130 is a computer or network of computers. Although a simplified example is illustrated in FIG. 2, typically the application server will be a server class system that uses powerful processors, large memory, and faster network components compared to a typical computing system used, for example, as a client device 110. The server typically has large secondary storage, for example, using a RAID (redundant array of independent disks) array and/or by establishing a relationship with an independent content delivery network (CDN) contracted to store, exchange and transmit data such as the asthma notifications contemplated above. Additionally, the computing system includes an operating system, for example, a UNIX operating system, LINUX operating system, or a WINDOWS operating system. The operating system manages the hardware and software resources of the application server 130 and also provides various services, for example, process management, input/output of data, management of peripheral devices, and so on. The operating system provides various functions for managing files stored on a device, for example, creating a new file, moving or copying files, transferring files to a remote system, and so on.

The application server 130 includes a software architecture for supporting access and use of the analytics system 100 by many different client devices 110 through network 150, and thus at a high level can be generally characterized as a cloud-based system. The application server 130 generally provides a platform for patients 111 and healthcare providers 112 to report data recorded by the sensors associated with their medicament devices 160 including both rescue medication and controller medication events, collaborate on respiratory ailment treatment plans, browse and obtain information relating to their condition and geographic location, and make use of a variety of other functions.

Generally, the application server 130 is designed to handle a wide variety of data. The application server 130 includes logical routines that perform a variety of functions including checking the validity of the incoming data, parsing and formatting the data if necessary, passing the processed data to a database server 140 for storage, and confirming that the database server 140 has been updated.

The application server 130 stores and manages data at least in part on a patient by patient basis. Towards this end, the application server 130 creates a patient profile for each user. The patient profile is a set of data that characterizes a patient 111 of the respiration ailment analytics system 100. The patient profile may include identity information about the patient such as age, gender, current rescue medication, current controller medication, notification preferences, a controller medication adherence plan, a relevant medical history, and a list of non-patient users authorized to access to the patient profile. The profile may further specify a device identifier, such as a unique media access control (MAC) address identifying the one or more client devices 110 or sensors 120 authorized to submit data (such as controller and rescue medication events) for the patient.

The profile may specify which different types of notifications are provided to patients 111 and their personal healthcare providers 112, as well as the frequency with which notifications are provided. For example, a patient 111 may authorize a healthcare provider 112 to receive notifications indicating a rescue event. The patient 111 may also authorize their healthcare provider 112 be given access to their patient profile and rescue event history. If the healthcare provider 112 is provided access to the patient profile of the patient 111, the healthcare provider may specify controller adherence or rescue medication plans for the corresponding COPD or asthma condition. Medication plans may include a prescribed number of doses per day for controller medications.

The application server 130 also creates profiles for healthcare providers 112. A health care provider profile may include identifying information about the healthcare provider 112, such as the office location, qualifications and certifications, and so on. The health care provider profile also includes information about their patient population. The provider profile may include access to all of the profiles of that provider's patients, as well as derived data from those profiles such as aggregate demographic information, rescue and controller medication event patterns, and so on. This data may be further subdivided according to any type of data stored in the patient profiles, such as by geographic area (e.g., neighborhood, city) over by time period (e.g., weekly, monthly, or yearly).

The application server 130 receives rescue medication event information from the client device 110 or the sensor 120, triggering a variety of routines on the application server 130. In the example implementations described below, the data analysis module 131 executes routines to access event data, sync data, as well as other data including a patient's profile, analyze the data, and output the results of its analysis to both patients 111 and providers 112. This process is generally referred to as a churn risk analysis. In this example, the churn risk analysis is performed in relation to use of a treatment device such as the inhaler 160. Alternatively, the churn risk may be performed in relation to a specific patient or a subgroup of patients.

Other types of analyses may include daily/weekly adherence trends, adherence changes over time, adherence comparisons to other relevant populations (e.g., all patients, patients on a particular rescue medication or controller medication or combination thereof, identification of triggers (spatial, temporal, environmental), rescue use trends over time, and rescue use comparisons to other relevant populations.

Responsive to any analyses performed, the application server 130 prepares and delivers push notifications to send to patients 111, authorized healthcare providers 112, and/or other users provided access to the patient's profile. Notifications can provide details about the timing, location, and affected patient(s) 111 involved in a medication rescue event. Notifications may additionally comprise a distress or emergency signal that requests emergency assistance that are distributed to emergency assistance providers 112. Notifications may also include the results of the churn analysis performed by the data analysis module 131. More information regarding the types of notifications that may be sent and the content they may contain is further described below.

In addition to providing push notifications in response to an asthma or COPD exacerbation risk analysis, notifications may also be provided as pull notifications, at particular time intervals. Additionally, some notifications (whether push or pull) may be triggered not in response to an asthma or COPD exacerbation risk analysis performed in response to a rescue medication event, but instead in response to a risk analysis performed in response to one of the underlying factors in the asthma or COPD exacerbation risk analysis changing, such that an updated notification is warranted. For example, if weather conditions indicate that an increase in air pollution is occurring or is imminent, this may trigger the carrying out of asthma exacerbation risk analyses for all patients located in the particular geographic area where the pollution is occurring.

Notifications are provided through the network 150 to client applications 115 in a data format specifically designed for use with the client applications, and additionally or alternatively may be provided as short message service (SMS) messages, emails, phone calls, or in other data formats communicated using other communication mediums.

The database server 140 stores patient and provider data related data such as profiles, medication events, patient medical history (e.g., electronic medical records). Patient and provider data are encrypted for security and is at least password protected and otherwise secured to meet all Health Insurance Portability and Accountability Act (HIPAA) requirements. Any analyses (e.g., asthma risk analyses) that incorporate data from multiple patients (e.g., aggregate rescue medication event data) and are provided to users is de-identified so that personally identifying information is removed to protect patient privacy. The database server 140 may also include customer service information in relation to the patient from the health care providers 112 such as inquiries, requests for assistance and the like.

The database server 140 may also store non-patient data. This data includes regional data about a number of geographic regions such as public spaces in residential or commercial zones where patients are physically located and may be exposed to pollutants. This data may specifically include or be processed to obtain a patient's proximity to green space (areas including concentrated numbers of trees and plants). One example of regional data includes georeferenced weather data, such as temperature, wind patterns, humidity, the air quality index, and so on. Another example is georeferenced pollution data, including particulate counts for various pollutants at an instance of time or measured empirically. The regional data includes information about the current weather conditions for the time and place of the rescue event such as temperature, humidity, air quality index. All of the items of data above may vary over time, and as such the data itself may be indexed by time, for example separate data points may be available by time of day (including by minute or hour), or over longer periods such as by day, week, month, or season. Although the database server 140 is illustrated in FIG. 1 as being an entity separate from the application server 130 the database server 140 may alternatively be a hardware component that is part of another server such as server 130, such that the database server 140 is implemented as one or more persistent storage devices, with the software application layer for interfacing with the stored data in the database is a part of that other server 130.

The database server 140 stores data according to defined database schemas. Typically, data storage schemas across different data sources vary significantly even when storing the same type of data including cloud application event logs and log metrics, due to implementation differences in the underlying database structure. The database server 140 may also store different types of data such as structured data, unstructured data, or semi-structured data. Data in the database server 140 may be associated with patients, groups of patients, and/or entities. The database server 140 provides support for database queries in a query language (e.g., SQL for relational databases, JSON NoSQL databases, etc.) for specifying instructions to manage database objects represented by the database server 140, read information from the database server 140, or write to the database server 140.

The network 150 represents the various wired and wireless communication pathways between the client devices 110, the sensor 120, the application server 130, and the database server 140. Network 150 uses standard Internet communications technologies and/or protocols. Thus, the network 150 can include links using technologies such as Ethernet, IEEE 802.11, integrated services digital network (ISDN), asynchronous transfer mode (ATM), etc. Similarly, the networking protocols used on the network 150 can include the transmission control protocol/Internet protocol (TCP/IP), the hypertext transport protocol (HTTP), the simple mail transfer protocol (SMTP), the file transfer protocol (FTP), etc. The data exchanged over the network 150 can be represented using technologies and/or formats including the hypertext markup language (HTML), the extensible markup language (XML), etc. In addition, all or some links can be encrypted using conventional encryption technologies such as the secure sockets layer (SSL), Secure HTTP (HTTPS) and/or virtual private networks (VPNs). In another embodiment, the entities can use custom and/or dedicated data communications technologies instead of, or in addition to, the ones described above.

FIG. 2 is a high-level block diagram illustrating physical components of an example computer 200 that may be used as part of a client device 110, application server 130, and/or database server 140 from FIG. 1, according to one embodiment. Illustrated is a chipset 210 coupled to at least one processor 205. Coupled to the chipset 210 is volatile memory 215, a network adapter 220, an input/output (I/O) device(s) 225, a storage device 230 representing a non-volatile memory, and a display 235. In one embodiment, the functionality of the chipset 210 is provided by a memory controller 211 and an I/O controller 212. In another embodiment, the memory 215 is coupled directly to the processor 205 instead of the chipset 210. In some embodiments, memory 215 includes high-speed random access memory (RAM), such as DRAM, SRAM, DDR RAM or other random access solid state memory devices.

The storage device 230 is any non-transitory computer-readable storage medium, such as a hard drive, compact disk read-only memory (CD-ROM), DVD, or a solid-state memory device. The memory 215 holds instructions and data used by the processor 205. The I/O device 225 may be a touch input surface (capacitive or otherwise), a mouse, track ball, or other type of pointing device, a keyboard, or another form of input device. The display 235 displays images and other information from the computer 200. The network adapter 220 couples the computer 200 to the network 150.

As is known in the art, a computer 200 can have different and/or other components than those shown in FIG. 2. In addition, the computer 200 can lack certain illustrated components. In one embodiment, a computer 200 acting as server 140 may lack a dedicated I/O device 225, and/or display 218. Moreover, the storage device 230 can be local and/or remote from the computer 200 (such as embodied within a storage area network (SAN)), and, in one embodiment, the storage device 230 is not a CD-ROM device or a DVD device.

Generally, the exact physical components used in a client device 110 will vary in size, power requirements, and performance from those used in the application server 130 and the database server 140. For example, client devices 110, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out the churn risk analyses introduced above. In one embodiment, the processing power of the application server 130 provided by a service such as Amazon Web Services™. Also, in contrast, the database server 140 may include many, physically separate computers each having a significant amount of persistent storage capacity for storing the data associated with the application server.

As is known in the art, the computer 200 is adapted to execute computer program modules for providing functionality described herein. A module can be implemented in hardware, firmware, and/or software. In one embodiment, program modules are stored on the storage device 230, loaded into the memory 215, and executed by the processor 205.

As an example of the process for capturing such an event for a particular device 160/sensor 120 combination, at the start of symptoms, the sensor 120 may detect whether a medicament dispenser 160 cover is opened. When the medication dispenser cover is opened, the sensor 120 may detect an acceleration associated with a priming of the dispenser 160. For some types of medicament dispensers, the “priming” includes activating a mechanism to release a dose of a medication from a packaging. In other types of medicament dispensers, the “priming” includes a rapid shaking of a medication canister.

After the priming action is detected, the sensor 120 is configured to store data associated with the rescue or control event in active memory of the sensor 120. The rescue or control event data may include information that describes the time and date of associated with the rescue event, the status or condition of the medicament device 160 (e.g. battery level), the number of doses of medication remaining (before or after the event), self-test results, and physiological data of a patient being treated with the medicament device 160 as measured by the sensor 120. As soon as the sensor establishes a network connection with either the client device 110 or network 150, the sensor transmits any locally stored rescue or control event data to the client device 110 or the application server 130. If the event data was transmitted to the client device 110 first, the client device 110 then transmits the rescue or control event data to the application server 130 as soon as the client device 110 establishes a network connection with the network 150. Depending upon the implementation, either the client device 110 or sensor 120 will add the geographic location where the event took place to the event data transmitted to the application server 130.

Upon receiving and storing the rescue usage event data, the application server 130 may request further information from the patient describing the rescue or control usage event. To obtain the information, the application server 130 generates a push notification, including the questions to be asked, to be sent to the patient's client device 110. The patient may respond to the request by providing inputs in response to the application. Alternatively, the patient 111 may elect not to respond to the request. This is permissible, any gaps in information may be obtained through later push notifications, or upon entry by provider 112 after a meeting with the patient 111. In one implementation, the failure to receive the additionally requested data in response to request does not hold up the remainder of the analysis.

In addition to requesting additional event data, the application server 130 accesses stored contextual data from the database server 140. Generally, contextual data refers data other than event data, which includes but is not limited to: to atmospheric conditions, weather conditions, air quality conditions, pollen data, patient data recorded from past rescue usage events, and any other considerations that are not directly detected by the medicament device at the time of the rescue usage event. By contrast, event data refers to any parameters related to the rescue event and reported by the medicament device, such as medication dosage, the time of the event, the location of the event, and relevant adherence data. Both forms of data may include temporally-current information as well as stored historical data. Accordingly, as part of obtaining the contextual data, the application server 130 also accesses historical rescue usage event data for the patient 111. This historical data can include all of the data from any past controller or rescue medication event data from the patient's history over a variety of windows of time in the past, and each historical event may include all of the same items of information as was reported for this event along with any data collected upon follow up thereafter.

During normal course, the database 140 receives data regarding rescue usage events as they occur and updates the primary patient data store and the general patient population data store to reflect the contextual data associated with the recent current rescue usage events for the primary patient and all the patients in the patient population. The frequency with which a data store is updated with new rescue usage events may vary depending on a number of factors including but not limited to the patient's circumstances, their adherence regimen to a controller medication, environmental conditions, and so on. A patient's adherence to a prescribed controller medication regimen is a comparison between the frequency per day that the patient uses the controller inhaler unit and the frequency per day that the patient was instructed to use the controller inhaler unit and may be measured based on the usage of a controller inhaler unit.

The system includes the application 115 executed on a portable computing device 110 that includes different interfaces. A data collection survey may be displayed to gather subjective patient data relating to the operation of the inhaler 160. The system stores the survey data entered by the patients.

In one example, the process for predicting churn for a treatment device or a patient is carried out by the data analysis module 131 of the analytic system 100 in FIG. 1 based on analysis of primary patient contextual data, patient reported outcomes, and other relevant data. The analytics system 100 includes, on the application server 130, a data analysis module 131 that analyses the variety of data collected by the system, introduced above and discussed further below, to predict churn rate for a patient or a device.

In this example, the analytics system based on the data analysis module 131 uses the Spark data processing engine via the AWS Glue extract, transform and load service to curate billions of disparate data points from sensors, phones, the application, service data relating to contacts to the health care provider or device manufacturer, and clinical/static patient data to create a comprehensive view of a patient at a daily level.

For purposes of the example predictive modeling, a churn event is defined as the first day of 30 consecutive days (or more) without a sensor synchronization with the application 115 on the mobile device 110 indicating the sensor 120 has not communicated with the application 115. This may be considered event data that indicates the patient is not using the medicament dispenser 160 because no sensor data can be received from the application. The synchronization allows the sensor 120 to collect additional data relating to use of the medicament inhaler 160. Different lengths of consecutive days may be selected. The goal of the example analysis system is to predict the probability of a churn event occurring within the next 30 days. Other periods of time other than 30 days may be used to predict the probability.

As explained above, the churn probability threshold may be set at any level to trigger an action such as a text message, email or push notification. Such a threshold will trigger an action any time a probability score exceeds that threshold. The accuracy of the trigger can be characterized in terms of churn event recall, defined as the proportion of events for which a trigger was created at least once in the prior 30 days before the churn events occur, and trigger/alert precision, defined as the proportion of triggers for which there was an event in the following 30 days. For example, the predictive engine in this example has a churn event recall of more than 75% and trigger/alert precision of more than 30%.

In this example, the production of the machine learning preferably includes best practices. These practices include full version control of data, models and code to enable reproducibility/analysis. Another best practice is rich job, model, variable and sample metadata to enable reproducibility/analysis. The performance monitoring is easily available from Glue tables produced by the extract, transform, and load (ETL) Glue service in this example. Retraining, retuning and variable changes are part of regular release cycles.

The machine learning model in this example uses Shapley additive explanations, the state of the art for model agnostic (in this case, gradient boosting trees) interpretability, to not only surface the characteristics or behaviors present in a patient over the past 30 days, but also what the impact of each individual behavior is on the probability of churn. These explanations can assist in better understanding the impact of behaviors both in aggregate across the population and at the patient-daily level. The technology stack (in addition to the data) in the example service may be used for future machine learning pipelines.

The data includes breadth of data sources, depth of the analytics and modularity to different scenarios. In this example, in relation to breadth of the sources, billions of data points are curated from the analytics system 100 into 50+ variables per patient day, and then further preprocessed into 500+ preprocessed behavioral variables summarizing the past 30 days. For example, a curated variable may be the “number of app opens today.” A preprocessed variable may be “the change in number of average app opens week on week”

In relation to depth, the primary response from the prediction endpoint contains the probability of churn in the next 30 days, the preprocessed input variables, and the impact of these preprocessed input variables. In relation to modularity the data sources, pipelines and the service itself each support multiple use cases. The data sources include causal analyses (data science) and behavioral dashboards (data analytics). The machine learning pipelines may predict any behavior such as application use or adherence with extremely limited code changes. Services include customer/patient service workflows, conditional marketing content, personalized product features and notifications.

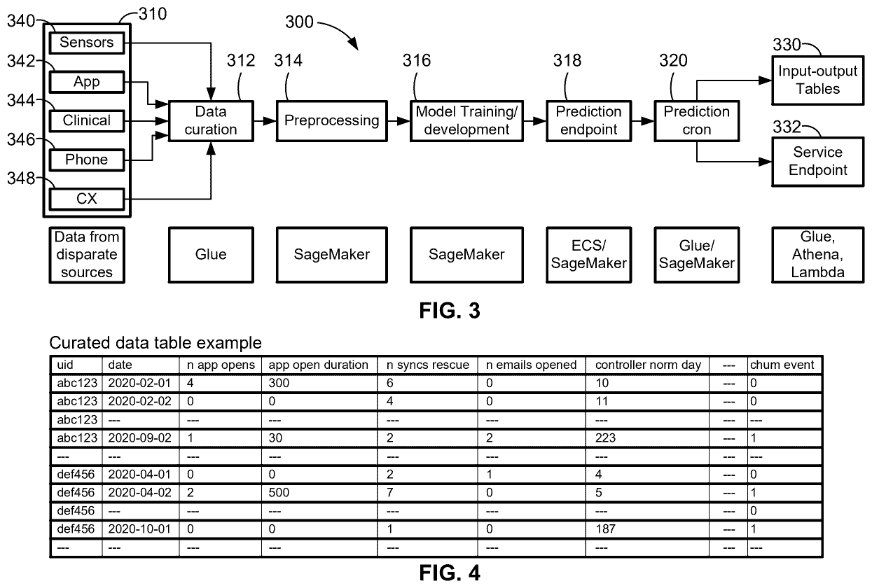

FIG. 3 shows an example machine learning pipeline 300. The machine learning pipeline includes a set of data inputs 310, a data curation module 312, a preprocessing module 314, a model training/development module 316, a prediction endpoint module 318, and a prediction cron module 320. The outputs include input/output tables 330 and a service endpoint 332. In this example, the data inputs 310 include sensor data inputs 340, application collected data inputs 342, clinical data inputs 344, mobile device data inputs 346, and customer service data inputs 348. In this example, the data curation module 312, prediction cron module 320 and outputs 330 and 332 are part of the Glue ETL. In this example, the preprocessing module 314, model training/development module 316, prediction endpoint module 318, and prediction cron 320 are part of the SageMaker service.

The data curation module 312 allows curation of data from different sources. Such sources may include but are not limited to a product backend, third party product analytics services, third party customer experience services, existing tables within a data lake, and existing tables within a data warehouse. These sources may be curated using multiple extract-transform-load (ETL) jobs involving parallel processing e.g. Apache Spark and AWS Glue. The final ETL job transforms all data from disparate sources into a single table where each row represents one day for one patient, and each column represents either metadata about that patient-day (e.g. a user ID or a date field) or a data point that summarizes a certain characteristic or behavior of a patient on that day.

In this example input data to the data curation module 312 includes the user ID and date as well as the data inputs 310. In this example, the sensor data inputs 340 are derived from event data from either the medicament inhaler 160 or the sensor 120 collected by the application. Examples of the sensor data points include: days since first rescue sensor sync with the application 115, days since first controller sensor sync with the application 114, number of syncs, number of sync retries, number of controller doses taken, controller adherence (proportion of prescribed doses taken), number of rescue uses, and whether or not that day contained a churn event (based on subsequent usage and sync patterns).

The application data inputs 342 are further analysis of event data and collected subjective data from the patient from the application 115. In this example, such inputs may include days since the application was first opened, number of application opens, duration of application opens, number of times different features were interacted with e.g. adding a trigger, viewing the trends tab, using the find my inhaler feature, duration of time different features were interacted with, number of responses to in-application feedback, number of push notifications received, and number of push notifications opened

The mobile device data inputs 346 are data derived from the mobile device such as time, location and movement of the patient. In this example, the data inputs 346 may include the operating system (i.e. Android, IOS, which version), the phone model, the percent of application heartbeats Bluetooth was enabled, the percent of application heartbeats location was enabled, and variance of location

The clinical data inputs 344 and customer service data inputs 348 may be derived from patient related health records and service records in the example database 140. In this example, the clinical data input may include disease, disease status, prescribed medications, doses, and clinical metrics such as a CAT or ACT score. In this example the customer service data inputs may include user interaction data with the provider 112, number of emails sent by the provider to the user, number of emails opened by user, and number of times user has interacted with support team e.g. via phone, email, text, chat.

FIG. 4 is an example curated data table produced by the example data curation module 312. The data table includes user IDs and dates in each row. Each column includes various events recorded for the particular user ID and date. For example, the first row includes the user ID and date with the number of application opens (4), the duration of the application being open (300 seconds), the number of syncs for a rescue inhaler medication sensor (8), the number of days since the first controller inhaler sync (10), and whether or not today represented a churn event (1 if true, 0 if false, in this case, 0).

The preprocessing module 314 in FIG. 3 consumes the final table from the data curation module 312, and further preprocesses the data such that it can be used directly by machine learning models for training, cross-validation and general development. This preprocessing includes but is not limited to: 1) the exclusion of any users that have not sufficiently interacted with the product or service (e.g. they have no sensor syncs or no app opens); 2) the inference of time variables from the date field (e.g. the year, month, and day of week); 3) the one-hot encoding of categorical variables into a binary array that can be interpreted by machine learning models; 4) the copying of certain variables to a model input table for which the only relevant data point is the current value (e.g. the number of days since the first sensor sync, the day of the week, the mobile platform); 5) the creation of input variables summarizing dynamic behavior over a defined trailing window (e.g. 30 days); 6) the creation of a binary target variable based on a predefined forward window (e.g. 30 days), and whether or not a churn event (defined as the first of at least 30 consecutive days of no sensor syncing) occurs within that window; and 7) the exclusion of any user-days according to rules.

The creation of input values summarizing dynamic behavior may include but not be limited to: a) the difference in moving average for combinations of 1, 2, 3, 7, 14, and 30 days; b) the difference in moving standard deviation for combinations of 1, 2, 3, 7, 14, and 30 days; c) the differences in sequential average (e.g. how the past 7 days vs. the prior 7 days before that) for combinations of 1, 2, 3, 7 and 14 days; d) the differences in sequential standard deviation for combinations of 1, 2, 3, 7 and 14 days; e) the average value per patient-day over 2, 3, 7, 14 and 30 days; and f) the standard deviation of values per patient-day over 2, 3, 7, 14 and 30 days.

The rules for excluding any user days may include but are not limited to: a) if the user-day is more than 30 days after the last churn event for that user; b) if there are any erroneous data points in the trailing or forward windows, e.g. null values or significantly outlying values; and c) if there is a user ID change in the forward or trailing window. The preprocessing module 314 then saves the final input and output arrays along with other relevant metadata into a data store such as an AWS S3 bucket.

The model training and development module 316 utilizes the data from the preprocessing module 314 and performs standard machine learning practices to develop a predictive model. The development may include, but not be limited to: 1) the assignment of each user-day sample into different cross-validation folds, partitioned based on user ID, date, or both (not random assignment of samples); 2) variable/feature selection based on heuristics or more complex methods including but not limited to correlation-based, information gain-based, cluster-based methods; 3) the training of machine learning models; 4) the selection of model hyperparameters and/or model class based on cross-validation performance; and 5) the generation of other relevant job data and metadata such as training time and predictive performance on each fold. The model training and development module 316 then saves a model artifact in a data store such as an AWS S3 bucket. This model artifact contains the model file itself along with all relevant job, sample and variable metadata.

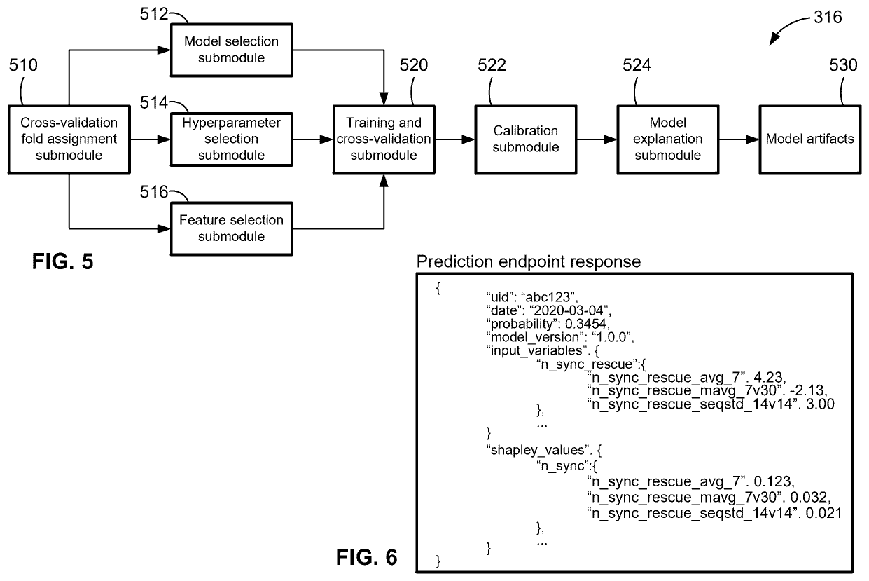

FIG. 5 is a block diagram of the model training and development module 316. The module 316 includes a cross validation fold assignment submodule 510, where each user-day sample is assigned a different grouping for purposes of cross-validation. The output of the submodule is fed into a model selection submodule 512 where a model class is chosen such as a gradient boosting tree, a random forest; a hyperparameter selection submodule 514, where hyperparameters are chosen, in this example including but not limited to the maximum depth, the learning rate, the split method, the strength of L1 or L2 regularization, the maximum number of leaves and the subsample ration; and 3) a feature selection submodule 516, where candidate input variables are selected based on a feature selection method such as a correlation-based method, an information gain-based method, or a cluster-based method. The outputs of the selection submodules 512, 514, and 516 are fed into a training and cross-validation submodule. 520. The output of the submodule 520 is fed into a calibration submodule 522, which corrects for any systematic overconfidence or underconfidence in prediction scores to ensure that such scores represent a true probability score. The output of the calibration submodule 522 is fed into a model explanation submodule 524, which performs analysis of the impact of each input variable in aggregate, for different user groups, for an individual user, or for an individual user-day, in this example by using Shapley additive values. The output is a set of model artifacts 530, storing all relevant information involved in the creation of the model.

The prediction endpoint module 318 accepts an input matching the schema of the final curated table, and containing data over the same trailing window defined by the preprocessing module 314. The endpoint may be in the form of preprocessing code and model artifacts produced by the model training development module 316 that can be consumed and used directly by a prediction cron job. It may also be in the form of an API where the input data over a trailing window can be posted to the endpoint, and the endpoint will preprocess the data in the same way as the preprocessing module, and respond with relevant data involved in making and understanding a predicted probability of churn.

Relevant data points that the prediction endpoint module 318 responds with include, but are not limited to: 1) the user ID; 2) the date; 3) the predicted probability or likelihood of a churn event in the defined forward window (e.g. 30 days); 4) the values of the processed input variables (e.g. current day values and trend values i.e. moving and sequential averages and standard deviations); and 5) the quantified impact of how these values influence the predicted probability, inferred using model explanation methods such as Shapley additive values. An example prediction endpoint response may be seen in FIG. 6. It is to be understood that the example pipeline modules are not limited to variables in FIG. 6 and there may be many other variables not shown in FIG. 6.

The prediction cron module 320 is executed at a predefined time and frequency (e.g. every day at 2 am), and iterates over each user's data from a curated table to extract the most recent trailing window of behavior defined in the preprocessing module. With this data, the prediction cron module 320 then interacts with the prediction endpoint to generate a predicted probability and other relevant information, and saves this information in one or more data stores such as AWS S3 buckets or AWS Glue tables.

The example input/output tables 330 may be the AWS S3 buckets or AWS Glue tables. Different stores or tables may serve different purposes. In this example, several tables may contain different information and serve different purposes including but not limited to: 1) a table where each row represents one user-day, with one column containing a user ID, one column containing the entire preprocessed input response as a string and one column containing the probability of churn; 2) a table where each row represents a specific preprocessed input value for a specific user-day, with one column containing a user ID, one column containing the date of prediction, one column containing the preprocessed input variable name, and one column containing the value; and 3) a table where each row represents a specific Shapley additive value for a specific user-day, with one column containing a user ID, one column containing the date of prediction, one column containing the preprocessed input variable name, and one column containing the Shapley additive value for that specific variable-user-day combination.

The service endpoint 332 makes the most recent information on each user's probability of churn available to other parts of the product or organization, including but not limited to customer experience products and services, and marketing automation tools to power, product personalization tools to customize the user experience. The service endpoint may take the form of direct queries on AWS Glue tables via another product such as AWS Athena. It may involve an API layer that allows an end user query for information including but not limited to: the probability a user ID will churn, other relevant data about a user ID's behavior and churn probability, all user IDs within a certain probability range, all user IDs within a certain percentile range of churn probability. These queries may contain other conditions for user IDs including but not limited to a specific program, region, disease, age range and clinical status.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

One or more elements or aspects or steps, or any portion(s) thereof, from one or more of any of claims 1-20 below can be combined with one or more elements or aspects or steps, or any portion(s) thereof, from one or more of any of the other claims 1-20 or combinations thereof, to form one or more additional implementations and/or claims of the present disclosure.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

What is claimed is:

1. A system to predict churn in relation to use of a treatment device by a patient, the system comprising:
a communication interface to collect event data on monitoring operation of the treatment device delivering medicament to the patient;
a storage device to store the event data and clinical data of the patient;
a churn analysis module operable to:
input the event data and clinical data;
apply a machine learning model to determine the likelihood of churn for the patient using the treatment device over a predetermined period from the input event data and clinical data, wherein the machine learning model was trained from a machine learning pipeline having inputs of event data and clinical data from a population of patients using treatment devices to determine at least one trigger for patient churn;
determining whether the likelihood of churn is over a threshold value; and
triggering an action relating to the patient if the likelihood of churn is over the threshold value.

2. The system of claim 1, wherein the treatment device is a rescue inhaler or a control inhaler.

3. The system of claim 1, wherein the communication interface communicates with a mobile device of the patient, and wherein a sensor in communication with the treatment device is synched with an application executed by the mobile device to provide event data.

4. The system of claim 1, wherein the machine learning pipeline is trained from customer service data relating to the population of patients.

5. The system of claim 1, wherein the action is one of a patient alert, a health care provider alert, or a treatment device manufacturer alert.

6. The system of claim 1, wherein the machine learning pipeline responds with a prediction endpoint including the determined likelihood of churn, the values of the processed clinical data and the event data, and a quantified impact of how the values influence the determined likelihood of churn.

7. A method to determine churn of a treatment device operated by a patient, the method comprising:
collecting clinical data of the patient;
collecting event data relating to monitoring operation of the treatment device via a communication interface;
applying a machine learning model to determine the likelihood of churn for the patient using the treatment device over a predetermined period from the input event data and clinical data, wherein the machine learning model was trained from a machine learning pipeline having inputs of event data and clinical data from a population of patients using treatment devices to determine at least one trigger for patient churn;
determining whether the likelihood of churn is over a threshold value; and
triggering an action relating to the patient if the likelihood of churn is over the threshold value.

8. The method of claim 7, wherein the treatment device is a rescue inhaler or a control inhaler.

9. The method of claim 7, further comprising:
communicating with a mobile device of the patient via the communication interface; and
synching a sensor in communication with the treatment device with an application executed by the mobile device to provide the event data.

10. The method of claim 7, wherein the machine learning pipeline is trained from customer service data relating to the population of patients.

11. The method of claim 7, wherein the action is one of a patient alert, a health care provider alert, or a treatment device manufacturer alert.

12. The method of claim 7, wherein the machine learning pipeline responds with a prediction endpoint including the determined likelihood of churn, the values of the processed clinical data and the event data, and a quantified impact of how the values influence the determined likelihood of churn.

13. A pipeline method of training a machine learning model for predicting the churn of a patient operating a treatment device, the method comprising:
collecting data inputs from a population of patients, the data inputs including clinical data and event data relating to monitoring operation of treatment devices;
curating the data inputs and producing a data table;
training the machine learning model from the data table to weight a plurality of input factors affecting churn and determine the impact of the input factors in determining a likelihood of churn to produce a prediction endpoint; and
storing the trained machine learning model.

14. The method of claim 13, further comprising:
refining the data table to exclude data relating to patients with insufficient interaction with the treatment device via a preprocessing module; and
converting data for input to the training module.

15. The method of claim 13, where the data inputs include data from mobile devices operated by the population of patients, data from an application executed on the mobile device, the application interfacing with the treatment device, and customer service data.

16. The method of claim 13, wherein the stored model is executed by a prediction endpoint module to provide a prediction of the likelihood of churn relating to a single patient based on clinical data and event data of the treatment device by the single patient.

17-20. (canceled)
